(19) United States
(12) Patent Application Publication
(10) Pub. No.: US 2012/0035744 A1
(43) Pub. Date: Feb. 9, 2012

(54) AMNION AND CHORION CONSTRUCTS AND USES THEREOF IN JOINT REPAIR

(75) Inventors: Robin R. YOUNG, Wayne, PA (US); Richard M. JAY, Philadelphia, PA (US)

(73) Assignee: AFCELL MEDICAL, Parsippany, NJ (US)

(21) Appl. No.: 13/198,330

(22) Filed: Aug. 4, 2011

Related U.S. Application Data
(60) Provisional application No. 61/370,593, filed on Aug. 4, 2010.

(51) Int. Cl. A61F 2/02

(52) U.S. Cl. 623/23.72

(57) ABSTRACT

A construct for use in a surgical repair of a damaged or inflamed joint is described. The construct contains an allograft having at least one layer of human amnion and chorion tissues, and is adapted for enclosing a damaged or inflamed joint or for covering or replacing a damaged or inflamed joint sheath during the surgical repair. Methods of preparing the construct and using it in a surgical repair of a damaged or inflamed joint are also described. The products and methods improve the performance of the joint surgery, e.g., by reducing inflammation, inhibiting fibrosis, scarring, fibroblast proliferation and post-operative infection while also promoting more rapid healing of damaged or traumatized joints.

### CROSS-REFERENCE TO RELATED APPLICATIONS

This application is entitled to priority pursuant to 35 U.S.C. §119 (e) to U.S. Provisional Patent Application No. 61/370.593, filed Aug. 4, 2010, which is hereby incorporated by reference herein in its entirety.

### BACKGROUND OF THE INVENTION

1. Field of Invention

Embodiments of the present invention relate to methods and products for improving the treatment of damaged or inflamed joints. In particular, embodiments of the present invention relate to constructs comprising an allograft having at least one layer of amnion and chorion tissues for use in surgical repair of damaged or inflamed joints to reduce inflammation, inhibit fibrosis, scarring, fibroblast proliferation, post-operative infection while also promoting more rapid healing of damaged or arthritic musculoskeletal joint structures.

2. Background of the Invention

The human musculoskeletal system relies on a system of joints to provide motion and flexibility to the skeletal frame. Musculoskeletal joints are complex internal structures composed of the ends of bones, ligaments, cartilage, an articular capsule and a synovial membrane that lines the capsule and secretes a lubricating fluid (synovia).

Various natural and traumatic events can cause the joints of the musculoskeletal system to deteriorate causing significant pain and reduced range of motion. The most common causes of joint deterioration are arthritis and direct physical trauma. Arthritis is the leading cause of disability in people over the age of 55 and there are several forms including osteoarthritis, rheumatoid arthritis, psoriatic arthritis. autoimmune diseases, septic arthritis and gouty arthritis.

Repairing damaged joints is hampered by several factors including damage to the protective sheathing which lines the capsule of each large joint and promotes smooth articulation. Despite numerous advances in surgical technique for repair of damaged joints, deterioration of an arthritic joint is considered by the medical community to he a chronic condition for which the rate of deterioration can be reduced but not stopped resulting in, for many patients, complete removal and replacement of the painful joint.

Therefore, it would be advantageous to have an implantable joint sheathing material that would effectively reduce joint inflammation and promote the natural healing process of the synovial membrane and articulating cartilage. Currently there is no allograft product available for use during surgical repair of damaged or severed large joints which would cover or replace synovial membrane that has been damaged by the arthritic process while also reducing pain and inflammation.

Human joints are among the internal structures that are covered by an extensive network of internal membranes. This protective network, generally called fascia, extends from the head to loot, front to hack of the human body and covers all internal structures. Depending on the structure, fascia may he thick or thin and performs several critical functions to maintain health of internal organs, large joints, tendons, spine, and nerves.

The protective membrane capsule that surrounds joints of the musculoskeletal system consists of several layers of loose connective collagen tissues. Inflammation of or damage to the synovial membrane causes harm to the large joint and will significantly limit the ability of the musculoskeletal system to ambulate.

A form of fascia that is similar to the synovial membrane is created during pregnancy to protect and facilitate the development of a fetus. This membrane is the amnion and its adjacent membrane, the chorion. These two inner linings of the placental sac surround and protect embryos in reptiles, birds, and mammals in a form of a capsule that is similar to the synovial capsule and other membrane sheathing and covers that protects all other internal structures of the human body including the joints.

The placental sac surrounds and covers the fetus and amniotic fluid or liquor amnii in a capsule not unlike the synovial membrane capsule which surrounds every musculoskeletal joint. The two inner layers of the placental sac are amnion and chorion which are tough, transparent, nerve-free. and nonvascular membranes consisting of multiple layers of cells: an inner, a thick layer of ectodermal epithelium and an outer covering of mesodermal, connective, and specialized smooth muscular tissue. In the later stages of pregnancy, the amnion expands to come in contact with the inner wall of the chorion creating the appearance of a thin wall of the sac extending from the margin of the placenta. The amnion and chorion are closely applied, though not fused, to one another and to the wall of the uterus. Thus, at the later stage of gestation, the fetal membranes are composed of two principal layers: the outer chorion that is in contact with maternal cells and the inner amnion that is bathed by amniotic fluid.

The amnion has multiple functions, i.e., as a covering epithelium, as an active secretary epithelium and for intense intercellular and transcellular transport. Before or during labor, the sac breaks and the fluid drains out. Typically, the remnants of the sac membranes are observed as the white fringe lining the inner cavity of the placenta expelled after birth. The amnion can be stripped off from the placenta. The amnion has a basement membrane side and a stroma side.

The fetal membrane including amnion and chorion has been used in surgeries documented as early as 1910. See Trelford et al., 1979, Am J Obstet Gynecol, 134:833-845. Amnioplastin, an isolated and chemically processed amniotic membrane, was used for continual dural repair, peripheral large joint injuries, conjunctival graft and flexor and large joint repair. See e.g., Chao et al., 1940, The British Medical Journal, March 30. The amnion has been used for multiple medical purposes, e.g., as a graft in surgical reconstruction forming artificial vaginas or over the surgical defect of total glossectomy, as a dressing for burns, on full-thickness skin wounds or in omphalocele, and in the prevention of meningocerebral adhesions following head injury or tissue adhesion in abdominal and pelvic surgery.

In recent years, there have been renewed interests in the application of amnion in ocular surface reconstruction, for example, as an allograph for repairing corneal defects. See, for example, Tsai and Tseng. Cornea. 1994 Scp;13(5):389-400: and Dua et al., Br. J. Ophthalmol 1999. 83:748-21) 752. In addition. amnion and amniotic fluid have recently been used as sources of placental stem cells. See. e.g.. U.S. Pat. No. 7,255,879 and WO 200073421.

There is a need of improved methods and products for improved surgical repair of damaged or inflamed joints to reduce inflammation, inhibit fibrosis, scarring, fibroblast proliferation. post-operative infection while also promoting more rapid healing of arthritic musculoskeletal joint structures. The present invention relates to such improved methods and products.

### BRIEF SUMMARY OF THE INVENTION

In one general aspect, embodiments of the present invention relate to a construct for use in a surgical repair of a damaged or inflamed joint. The construct comprises an allograft comprising at least one layer of human amnion and chorion tissues, wherein the construct has a shape adapted for enclosing a damaged or inflamed joint or for covering or replacing a damaged or inflamed joint sheath during the surgical repair.

In another general aspect. embodiments of the present invention relate to a method of preparing a construct for use in a surgical repair of a damaged or inflamed joint. The method comprises drying an allograft comprising at least one layer of amnion and chorion tissues on a frame, preferably a rigid or semi-rigid frame, of a shape appropriate for enclosing a damaged or inflamed joint or for covering or replacing a damaged or inflamed joint sheath, such as a damaged or inflamed synovial membrane, during the surgical repair.

Another aspect of the present invention relates to a method of performing a surgical repair of a damaged or inflamed joint in a subject. The method comprises:

(a) surgically repairing the damaged or inflamed joint to obtain a surgically repaired joint in the subject; and

(b) covering the surgically repaired joint or a damaged or inflamed joint sheath with at least one of an amniotic fluid and a construct, or replacing the damaged or inflamed joint sheath with the construct prior to wound closing,

wherein the construct comprises at least one layer of human amnion and chorion tissues, and the construct has a shape adapted for enclosing the surgically repaired joint or for covering or replacing the damaged or inflamed joint sheath.

Yet another general aspect of the present invention relates to a kit, which comprises:

(a) a construct for use in a surgical repair of a damaged or inflamed joint; and

(b) instructions on how to use the construct in the surgical repair,

wherein the construct comprises an allograft comprising at least one layer of human amnion and chorion tissues, and the construct has a shape adapted for enclosing a damaged or inflamed joint or for covering or replacing a damaged or inflamed joint sheath during the surgical repair.

In a preferred embodiment of the present invention, the human amnion and chorion tissues used in the present invention are obtained by a process comprising:

(a) obtaining informed consent from pregnant females;

(b) conducting risk assessment on the consented pregnant females to select an amnion donor;

(c) procuring after birth placenta from the amnion donor; and

(d) obtaining the human amnion and chorion tissues from the placenta.

Other aspects, features and advantages of the invention will be apparent from the following disclosure, including the detailed description of the invention and its preferred embodiments and the appended claims.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawing:

FIG. 1 illustrates a construct for use in a surgical repair of a damaged or inflamed joint according to an embodiment of the present invention.

### DETAILED DESCRIPTION OF THE INVENTION

Unless defined otherwise, all technical and scientific terms used herein have the same meaning as commonly understood to one of ordinary skill in the art to which this invention pertains. In this application, certain terms are used, which shall have the meanings as set in the specification. It must he noted that as used herein and in the appended claims the singular forms “a,” “an,” and “the” include plural reference unless the context clearly dictates otherwise.

Embodiments of the present invention relate to an amnion and/or chorion construct for use in a surgical repair of a damaged or inflamed joint. The construct comprises an allograft comprising at least one layer of human amnion and chorion tissues. The construct is made into a shape and thickness uniquely designed for the surgical repair of a damaged or inflamed joint, e.g., for enclosing one or more damaged or inflamed joints or for covering or replacing a damaged or inflamed joint sheath, such as a damaged or inflamed synovial membrane, during the surgical repair. The construct can be prepared by drying an allograft with the required shape, optionally over a frame. The frame can be flexible, but preferably rigid or semi-rigid. The frame can be a resorbable frame, e.g., polymer mesh frame, or a disposable or stainless steel frame, of the adequate shape.

Such shape can be, for example, concave bowls or curved sheets. The configuration of the construct is uniquely designed to allow for ease of application in a joint repair surgery to thereby facilitate and improve surgical repair of damaged or inflamed joints. The construct can be sutured or anchored onto bones or surfaces in the joint. It can also be delivered to the patient via minimally invasive surgery techniques.

There are different types of joints classified by structures or functions. A synovial joint is the most common and most movable type of joint in the body of a mammal. In a normal synovial joint in the knee, the articulating surfaces of a synovial joint is surrounded by an envelope, a fibrous joint capsule continuous with the periosteum of bone. The inner layer of the capsule is the synovial membrane, a soft tissue that lines the non-cartilaginous surfaces within joints with cavities.

Lubricating synovial fluid is filled within the cavities. The bones at the joint are covered with cartilage. a stiff and inflexible connective tissue composed of specialized cells, i.e., chondrocytes, which produce a large amount of extracellular matrix composed of collagen fibers, abundant ground substance rich in proteoglycan, and elastin fibers.

A synovial joint in the hip has similar structural components as those in the knee joint, including, e.g., the joint capsule, synovial membrane, synovial fluid and cartilage.

A damaged joint can be caused by injury, inflammation, or other diseases or disorders of any component of the joint. For example, osteoarthritis (degenerative joint disease), the most common form of arthritis, can be caused by trauma to the joint, infection of the joint, or aging. A knee joint having osteoarthritis can also be caused by thinned cartilage. Damaged cartilage is difficult to heal, because it has limited repair capabilities. As a result, the bone ends rub together in the joint, causing severe pain.

Rheumatoid arthritis, which affects about 1% of the world's population, is a chronic, systemic inflammatory disorder principally affecting synovial joints. A knee joint having rheumatoid arthritis can have a swollen inflamed synovial membrane and bone erosion. Joints affected with rheumatoid arthritis are painful and stiff.

Surgeries are required to repair some damaged joints. For example, complete or partial joint replacement surgery has been used to replace an arthritic or dysfunctional joint surface with an orthopaedic prosthesis to treat damaged joints. Joint replacement surgery has been used to treat, for example, osteoarthritis, rheumatoid arthritis, avascular necrosis or osteonecrosis, congenital dislocation oldie hip joint, hip dysplasia, acetabular dysplasia (shallow hip socket). frozen shoulder & loose shoulder, traumatized and malaligned joint, or joint stiffness. In a joint replacement surgery, usually the diseased or damaged joint surfaces are replaced With metal and/or plastic components shaped to allow continued motion of the joint.

One general aspect of the present invention relates to a method of performing a surgical repair of a damaged or inflamed joint in a subject. The method comprises: (a) surgically repairing the damaged or inflamed joint to obtain a surgically repaired joint in the subject; and (b) covering the surgically repaired joint or a damaged or inflamed joint sheath with at least one of an amniotic fluid and a construct, or replacing the damaged or inflamed joint sheath with the construct, prior to wound closing, wherein the construct comprises at least one layer of human amnion and chorion tissues, and the construct has a shape adapted for enclosing the surgically repaired joint or for covering or replacing the damaged or inflamed joint sheath.

The amniotic fluid and the construct can he applied individually or in combination during the surgery. The damaged or inflamed joint sheath can be associated With the damaged or inflamed joint. The damaged joint sheath can also be resulting from the surgical repair of the damaged or inflamed joint. Preferably, the amniotic fluid is processed so that it has a relatively high viscosity for ease of application and for remaining in the desired area after the application. Methods known to those skilled in the art can be used to prepare an amniotic fluid with a relatively high viscosity in view of the present disclosure.

In one embodiment of the present invention, both the amniotic fluid and the replacement cover are applied to cover the surgically repaired joint or damaged or inflamed joint sheath during the surgery. Preferably, the amniotic fluid has a relatively high viscosity.

In another embodiment of the present invention, only the amniotic fluid, preferably, an amniotic fluid with a relatively high viscosity, is applied to cover the surgically repaired joint or the damaged or inflamed joint sheath, prior to wound closing.

In another embodiment of the present invention, only the construct is applied to cover the surgically repaired joint or damaged or inflamed joint sheath or to replace the damaged or inflamed joint sheath, prior to wound closing.

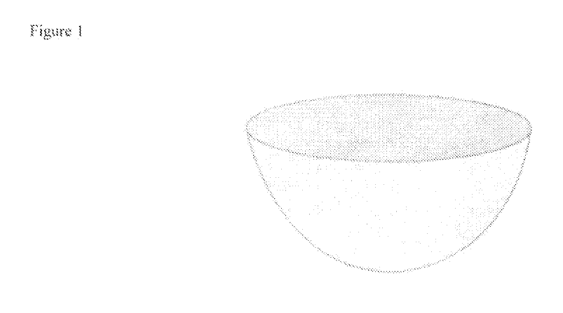

According to an embodiment of the present invention, a construct comprising an allograft having at least one layer of amnion and chorion tissues is used to enclose one or more damaged or inflamed joints or to cover or replace a damaged or inflamed joint sheath, such as a damaged or inflamed synovial membrane, during a surgical repair. The construct is of a shape and thickness suitable for enclosing the one or more damaged or inflamed joints or covering or replacing the damaged or inflamed joint sheath during the surgical repair. FIG. 1 illustrates a construct of a concave bowl or curved sheet shape that can be used in the present invention. Other shapes of construct having the size and characteristics similar to those of a synovial capsule can also be used in view of the present disclosure.

In one embodiment of the present invention, the construct further comprises a frame, which can he rigid, semi rigid or flexible, preferably the frame is rigid and semi rigid. The frame can he disposable or implantable and resorbable.

In another embodiment of the present invention, one or more corners of the construct are rounded or flatted to prevent the corners from catching during implantation. In view of the present disclosure, any method known to those skilled in the art can be used to make the corners of the construct round or flatten.

The allograft amnion and/or chorion is processed in such a way as to become a rigid or semi-rigid shape which is appropriate for the surgical need. Whichever form is used, the resulting allograft tissue retains its shape up to and including implantation into the patient. Following implantation and when the tissue is hydrated, the shape can relax and the implanted membrane can conform to the damaged joint surfaces.

The membrane shapes can be of various lengths and diameters to fit the various joint structures in the body. For example, the thickness of the allograft comprising amnion and/or chorion can he between 0.25 mm to 2.0 mm.

According to an embodiment of the present invention, a construct according to an embodiment of the present invention can be used in a complete or partial joint replacement surgery, for example. to cover the replaced joint, or to cover or replace the damaged or inflamed joint sheath resulting from the joint replacement surgery. prior to wound closing, to thereby reduce adhesion and inflammation.

In another embodiment of the present invention, a construct comprising an allograft having at least one layer of human amnion and chorion tissues is used to cover a skin incision resulting from the surgery. The allograft can he of any size suitable for covering the sutures or other type of tissue injuries at skin incision. An amniotic fluid, preferably, an amniotic fluid with a relatively high viscosity, can also be used to cover the skin incision, alone or in combination with the construct.

Preferably, a relatively thick layer of allograft is used to cover the skin incision. In one embodiment of the invention, the allograft patch has a thickness of about 2 mm to 4 mm. It can have multiple layers of amnion or a combination of multiple layers of amnion and chorion in any combination of amnion and chorion.

There are several attributes which make amnion a preferred material for protecting large joints which have been surgically repaired with sutures or other connective devices. Amnion has a complete lack of surface antigens and therefore does not induce an immune response when implanted into a ‘foreign’ body, which is in contrast to most other implants. Amnion also markedly suppresses the expression of the pro-inflammatory cytokines, IL-1α and IL-1β (Solomon et al., 2001. Br J Ophthalmol. 85(4):444-9) and produces natural inhibitors of matrix 20 metalloproteases (MMPs) expressed by infiltrating polymorphonuclear cells and macrophages. Hao et al., 2000, Cornea, 19(3):348-52: Kim et al., 2000, Exp Eve Res. 70(3):329-37). Amnion also down-regulates TGF-β and its receptor expression by fibroblasts leading to the ability to modulate the healing of a wound by promoting tissue reconstruction. Furthermore, amnion and chorion contain antimicrobial compounds with broad spectrum activity against bacteria, fungi, protozoa, and viruses for reduced risk of post-operative infection. All of these characteristics of amnion make it a potential allograft candidate to be used in treating damaged or inflamed joints.

Human allograft amnion and chorion have the ability to reduce inflammation, inhibit microbial infection and improve healing. Repairing damaged or inflamed joints, such as arthritic large joints, requires the surgeon to work in very tight spaces, and recovering the synovial capsule is extremely difficult. Surgeons who would attempt to recover the synovial capsule could encounter several problems.

By creating a curved shape, preferably a rigid or semi-rigid curved shape, which mimics the size and characteristics of a human synovial capsule, the present invention improves the ability of the surgeon to cover or replace damaged synovial membranes and thereby reduces inflammation. promotes healing and possibly delays or eliminates the need for removal and replacement of the arthritic joint.

Amnion tissues used in the present invention can be prepared from birth tissue procured from a pregnant female. Informed consent is obtained from a pregnant female by following guidelines as promulgated by the American Association of Tissue Banks and consistent with guidelines provided the Food and Drug Administration: a federal agency in the Department of Health and Human Services established to regulate the release of new medical products and, finally, if required by an established review body of the participating hospitals or institutions. The pregnant female is informed that she will be subject to risk assessment to determine if she is qualified as a birth tissue donor. She will also be informed of the tests for the risk assessment. The pregnant female is further informed that, if she is selected as a birth tissue donor based on the risk assessment, her birth tissues, such as placenta and amniotic fluid, may be collected at birth, tested and processed tor medical uses.

The informed consent includes consent for risk assessment and consent for donation of birth tissues.

Risk assessment is conducted on a pregnant female with informed consent to evaluate her risk factors for communicable diseases, such as human immunodeficiency virus (HIV). hepatitis B virus (HBV). hepatitis C virus (HCV). cytomegalovirus (CMV), human T-lymphotropic virus (HTLV), syphilis, etc. Medical and social histories of the pregnant female, including physical exam record, and/or risk assessment questionnaire, are reviewed. Pregnant females with high risk factors for the communicable diseases are excluded.

Consent to draw blood at time of delivery and 1 to 12 months post delivery is obtained from pregnant females with low risk factors for the communicable diseases. Screening tests on communicable diseases, such as HIV 1 and 2. HCV, Hb Core, syphilis, HTLV I/II, CMV, hepatitis B and C, are conducted by conventional serological tests on the blood sample obtained at birth. The initial screening tests are preferably completed within 7 days after birth. Preferably, the screening tests are conducted again on a second blood sample collected a few months post delivery, to verify the previous screening results and to allow for detection of communicable disease acquired shortly before birth, but are shown as “negative” on the previous screening tests. The second blood sample can be collected 1-12 months, preferably 6 months, post birth.

Only pregnant females with informed consent who arc tested negative for the communicable diseases are approved as birth tissue donor. In a preferred embodiment, only pregnant females with informed consent who are tested negative for the communicable diseases in both screening tests with the blood sample drawn at birth and the blood sample drawn 6 months post delivery are approved as birth tissue donor.

Sterile techniques and procedures should he used as much as practically possible in tissue handling, e.g., during tissue procurement, banking, transfer, etc., to prevent contamination of the collected tissues by exogenous pathogens.

Only birth tissues procured from the approved birth tissue donors are subject to the collection and subsequent processing. Birth tissues, such as placenta and amniotic fluid, are recovered from the delivery room and are transferred to a location in a sterile container, such as a sterile plastic bag or bottle. Preferably, the tissues are transferred in a thermally insulated device at a temperature of 4° to 28° C. for example, in an ice bucket.

According to an embodiment of the invention, shortly after its expulsion after birth, a suitable human placenta is placed in a sterile zip-lock plastic bag. which is placed in an ice bucket, and is delivered to another location. The placenta is rinsed, e.g., with sterile saline, to removed excessive blood clots. Preferably, the placenta is subject to aseptic processing, for example, by including one or more antibiotics, such as penicillin and/or streptomycin, in the rinse. The aseptically processed placenta is stored in a controlled environment, such as hypothermic conditions, to prevent or inhibit apoptosis and contamination.

The processed placenta is placed in a sterile container, such as one made of triple sterile plastic bags, packed in wet ice, and shipped to a location for subsequent processing via overnight courier. The placenta is shipped together with release documents for processing. For example, each shipment must include technical approval to process based upon a satisfactory review of the criteria for donor selection and donor approval. The shipment must also include results on screening of communicable diseases. Preferably, the shipment includes medical director review and approval of donor eligibility/suitability.

Upon receiving the shipment and a satisfactory review of the accompanying release documents, the amnion is separated from the chorion and other remaining tissues of placenta using methods known in the art in view of the present disclosure. For example, the amnion can be stripped off mechanically from the placenta immersed in an aseptic solution, e.g., by tweezers. The isolated amnion can be stored in a cryoprotective solution comprising a cryoprotective agent, such as dimethyl sulfoxide (DMSO) and glycerol, and cryopreserved by using a rapid, flash-freeze method or by controlled rate-freeze methods. Preferably, the isolated amnion is treated with one or more antibiotics, such as penicillin and/or streptomycin, prior to cryopreservation. The chorion can also he separated from the other tissues. preserved and stored for future use.

The isolated amnion is a tough, transparent. nerve-free and nonvascular sheet of membrane. It can be dried or lyophilized using various methods. For example. it can be dried over a sterile mesh, for example, by being placed on a sterile nitrocellulose filter paper and air dried for more than 50 minutes in a sterile environment. It can also be dried or lyophilized over other form of supporting material. which would facilitate the subsequent manipulation of the amnion, such as sterilizing. sizing, cataloging, and shipping of the amnion.

The present invention encompasses a kit comprising a construct for use in a surgical repair of a damaged or inflamed joint and instructions on how to use the construct in the surgery. Any of the constructs for use in a joint surgery according to embodiments of the present invention can he included in the kit. The construct comprises an allograft comprising at least one layer of human amnion and chorion tissues. The construct is adapted for enclosing one or more damaged or inflamed joint or for covering or replacing a damaged or inflamed joint sheath. such as a damaged or inflamed synovial membrane, during the surgical repair. The kit can further comprise amniotic fluid and instructions on how to use the amniotic fluid in the surgical repair of a damaged or inflamed joint. Preferably, the amniotic fluid has a relatively high viscosity.

In a preferred embodiment. the kit comprises a plurality of constructs for the joint surgery. and at least two of the plurality of constructs have different shapes or sizes suitable for covering different surgical sites. The construct and/or the amnion fluid can further comprise one or more therapeutically active agents. such as anti-microbial agents. growth enhancing agents. anti-inflammatory agents, analgesics, etc., for further improvement of the performance and reduction of the complications of the surgical repair.

It will be appreciated by those skilled in the art that changes could be made to the embodiments described above without departing from the broad inventive concept thereof. It is understood. therefore. that this invention is not limited to the particular embodiments disclosed, but it is intended to cover modifications within the spirit and scope of the present invention as defined by the appended claims.

1. A construct for use in a surgical repair of a damaged or inflamed joint, the construct comprising an allograft comprising at least one layer of human amnion and chorion tissues, wherein the construct has a shape adapted for enclosing a damaged or inflamed joint or for covering or replacing a damaged or inflamed joint sheath during the surgical repair.

2. The construct of claim 1. further comprising a frame that is disposable or implantable and resorbable.

3. The construct of claim 2. wherein the frame is rigid or semi rigid.

4. The construct of claim 1, wherein the shape is concave bowl or curved sheet.

5. The construct of claim 1, having one or more rounded or flattened corners.

6. The construct of claim 1. wherein the joint is a synovial joint and the joint sheath is a synovial membrane.

7. The construct of claim 1, further comprising one or more therapeutic agents.

8. The construct of claim 1, wherein the human amnion and chorion tissues are obtained using a process comprising:
a. obtaining informed consent from pregnant females:
b. conducting risk assessment on the consented pregnant females to select an amnion donor;
c. procuring after birth placenta from the amnion donor; and
d. obtaining the human amnion and chorion tissues from the placenta.

9. A method of preparing a construct of claim 1 the method comprising drying the allograft on the frame.

10. A method of performing a surgical repair of a damaged or inflamed joint in a subject, comprising:
a. surgically repairing the damaged or inflamed joint to obtain a surgically repaired joint in the subject: and
b. covering the surgically repaired joint or a damaged or inflamed joint sheath with at least one of an amniotic fluid and a construct, or replacing the damaged or inflamed joint sheath with the construct, prior to wound closing,
wherein the construct comprises at least one layer of human amnion and chorion tissues, and the construct has a shape adapted for enclosing the surgically repaired joint or for covering or replacing the damaged or inflamed joint sheath.

11. The method of claim 10, further comprising applying an allograft comprising at least one layer of human amnion and chorion tissues over a suture line or an incision resulting from the surgical repair to form a cover and barrier over the suture line or the incision..

12. The method of claim 10, wherein the construct further comprises a rigid or semi rigid frame.

13. The method of claim 10, wherein the joint is a synovial joint and the joint sheath is a synovial membrane.

14. The method of claim 10. wherein the construct has one or more rounded or flattened corners.

15. The method of claim 10, wherein the construct further comprises one or more therapeutic agents.

16. The method of claim 10, wherein the amniotic fluid and the human amnion and chorion tissues are obtained using a process comprising:
a. obtaining informed consent from pregnant females;
b. conducting risk assessment on the consented pregnant females to select an amnion donor:
c. procuring the amniotic fluid from the amnion donor;
d. procuring alter birth placenta from the amnion donor; and
e. obtaining the human amnion and chorion tissues from the placenta.

17. The method of claim 10. wherein the surgical repair is a complete or partial joint replacement surgery.

18. A kit comprising:
(a) a construct for use in a surgical repair of a damaged or inflamed joint; and
(b) instructions on how to use the construct in the surgical repair.
wherein the construct comprises an allograft comprising at least one layer of human amnion and chorion tissues. and the construct has a shape adapted for enclosing the damaged or inflamed joint or for covering or replacing a damaged or inflamed joint sheath during the surgical repair.

19. The kit of claim 18, comprising a plurality of constructs for use in the surgical repair of the damaged or inflamed joint. wherein at least two of the plurality of'constructs have different shapes or sizes.

20. The kit of claim 19, further comprising an amniotic fluid and instructions on how to use the amniotic fluid in the surgical repair.
